(19) United States
(12) Patent Application Publication
(10) Pub. No.: US 2024/0236567 A9
(43) Pub. Date: Jul. 11, 2024

(54) ADAPTIVE AUDIO SYSTEM FOR OCCUPANT AWARE VEHICLES

(71) Applicant: QUALCOMM Incorporated, San Diego, CA (US)

(72) Inventors: Asif Mohammad, San Diego, CA (US); Laxmi Rayapudi, San Diego, CA (US); Eddie Choy, Carlsbad, CA (US)

(21) Appl. No.: 18/470,800

(22) Filed: Sep. 20, 2023

Related U.S. Application Data
(60) Provisional application No. 63/380,168, filed on Oct. 19, 2022.

(51) Int. Cl. H04R 3/12; A61B 5/00; B60H 1/00; G10K 11/16

(52) U.S. Cl. CPC H04R 3/12; A61B 5/4809; B60H 1/00742; G10K 11/16; H04R 2499/13

(57) ABSTRACT

In general, this disclosure describes various aspects of the techniques directed to an adaptive audio system for occupant aware vehicles. A device configured to reproduce a soundfield based on audio data within a vehicle may implement the techniques. The device may comprise a memory configured to store the audio data, and processing circuitry coupled to the memory. The processing circuitry may be configured to obtain, from an occupant monitoring system, a state of an occupant residing within a cabin of the vehicle, modify, based on the state of the occupant residing within the cabin of the vehicle, playback of the audio data within at least a portion of the cabin of the vehicle to obtain modified playback data for the audio data, and reproduce, based on the modified playback data, the soundfield.

This application claims the benefit of U.S. Provisional Application Ser. No. 63/380,168, entitled “ADAPTIVE AUDIO SYSTEMS FOR OCCUPANT AWARE VEHICLES,” filed Oct. 19, 2022, the entire contents of which are hereby incorporated by reference.

### TECHNICAL FIELD

This disclosure relates to processing of media data, such as audio data.

### BACKGROUND

Many vehicles are equipped occupant monitoring systems (OMS) that provide information regarding occupants in the vehicle, such as an operator of the vehicle to determine awareness of a contextual operating environment in which the operator is operating the vehicle (e.g., a state of the operator—such as impaired, asleep, distracted, etc.), passengers of the vehicle to monitor presence (e.g., for deploying airbags), safety (e.g., notifying of status of the seatbelt in an dashboard notification system), etc. Such vehicles may also be equipped with entertainment or infotainment systems, which reproduce a soundfield, based on audio data (or in other words, audio signals), via loudspeakers.

### SUMMARY

This disclosure relates generally to an adaptive audio system for occupant aware vehicles. A vehicle head unit or other device may implement the adaptive audio system, interfacing with the occupant monitoring system (OMS) to receive status updates regarding occupants residing within a cabin of the vehicle. The adaptive audio system may receive status updates indicating that, as an example, a rear passenger residing within a rear passenger zone of the cabin of the vehicle is sleeping (e.g., a baby is sleeping). Rather than fully reproduce the soundfield throughout the vehicle, the adaptive audio system may modify playback of the audio data based on the status updated provided by the OMS (where, in this example, the adaptive audio system may mute the playback of audio data in the rear-passenger zone of the cabin of the vehicle).

In this respect, various aspects of the techniques may improve operation of the infotainment system (which is another way to refer to a vehicle head unit) itself. For example, rather than indiscriminately reproduce a soundfield in all portions of the cabin of the vehicle, the adaptive audio system may modify (or, in other words, adapt) audio playback to mute reproduction of the soundfield in one or more portions of the cabin of the vehicle (or, in other words, the vehicle cabin) based on status updates provided by the OMS. Muting the volume of playback in specific portions of the vehicle cabin may maintain, facilitate, or support continuation of the status of occupants (e.g., sleeping children) monitored by the OMS. By facilitating continuation of the status of the occupants, other passengers of the vehicle may continue to enjoy content (e.g., playback of the audio data) without distractions or otherwise interfering with the status of other occupants within the vehicle cabin, thereby improving enjoyment by occupants of the content reproduction provided by the infotainment system itself.

In one example, various aspects of the techniques are directed to a device configured to reproduce a soundfield based on audio data within a vehicle, the device comprising: a memory configured to store the audio data; and processing circuitry coupled to the memory, and configured to: obtain, from an occupant monitoring system, a state of an occupant residing within a cabin of the vehicle; modify, based on the state of the occupant residing within the cabin of the vehicle, playback of the audio data within at least a portion of the cabin of the vehicle to obtain modified playback data for the audio data; and reproduce, based on the modified playback data, the soundfield.

In another example, various aspects of the techniques are directed to a method of reproducing a soundfield based on audio data within a vehicle, the method comprising: obtaining, from an occupant monitoring system, a state of an occupant residing within a cabin of the vehicle; modifying, based on the state of the occupant residing within a cabin of the vehicle, playback of the audio data within at least a portion of the cabin of the vehicle to obtain modified playback data for the audio data; and reproducing, based on the modified playback data, the soundfield.

In another example, various aspects of the techniques are directed to a non-transitory computer-readable storage medium having instructions stored thereon that, when executed, cause one or more processors of a vehicle headunit to: obtain, from an occupant monitoring system, a state of an occupant residing within a cabin of a vehicle including the vehicle headunit; modify, based on the state of the occupant residing within the cabin of the vehicle, playback of audio data representative of a soundfield within at least a portion of the cabin of the vehicle to obtain modified playback data for the audio data; and reproducing, based on the modified playback data and the audio data, the soundfield.

The details of one or more examples of this disclosure are set forth in the accompanying drawings and the description below. Other features, objects, and advantages of various aspects of the techniques will be apparent from the description and drawings, and from the claims.

### BRIEF DESCRIPTION OF DRAWINGS

FIG. 1 is a block diagram illustrating an example vehicle configured to perform various aspects of the adaptive audio system techniques described in this disclosure.

FIGS. 2A-2D are diagrams illustrating example operation of the AAS shown in the example of FIG. 1 in perform various aspects of the adaptive audio playback techniques described in this disclosure.

FIG. 3 is a flowchart illustrating example operation of the vehicle shown in the example of FIG. 1 in performing various aspects of the adaptive audio playback techniques described in this disclosure.

FIG. 4 is a conceptual diagram illustrating an example of a wireless communications system in accordance with aspects of the present disclosure.

### DETAILED DESCRIPTION

Many vehicles are equipped with entertainment or infotainment systems (which is another way to refer to a vehicle headunit), which reproduce a soundfield, based on audio data (or in other words, audio signals), via loudspeakers. In addition, many vehicles, such as the vehicle 100, include an occupant monitoring system (OMS) that may monitor a status of occupants within the cabin of the vehicle.

The infotainment system may be configured to adapt operating conditions (e.g., present safety notifications, such as the status of the seatbelt for a passenger) dependent upon status updates provided by the OMS. That is, the infotainment systems may use status updates provided by the OMS to adapt user interfaces, activate one or more of the cameras, activate one or more of the microphones, etc. to enable occupant aware functionality regarding safety, awareness, activity, etc. occurring within the cabin of the vehicle. However, the infotainment systems may not adapt reproduction of a soundfield based on OMS status updates concerning the various occupants within the vehicle.

In accordance with various aspects of the techniques described in this disclosure, a vehicle or other device (e.g., a vehicle head unit) may implement an adaptive audio system (AAS), interfacing with the OMS to receive status updates regarding occupants residing within a cabin of the vehicle. The adaptive audio system may receive status updates indicating that, as an example, a rear passenger residing within a rear passenger zone of the cabin of the vehicle is sleeping (e.g., a child—baby—is sleeping). Rather than fully reproduce the soundfield throughout the vehicle, the adaptive audio system may modify playback of the audio data (AD) based on the status updated provided by the OMS (where, in this example, the adaptive audio system may mute the playback of the audio data in the rear-passenger zone of the cabin of the vehicle).

In this respect, various aspects of the techniques may improve operation of the infotainment system (which is another way to refer to a vehicle head unit) itself. For example, rather than indiscriminately reproduce a soundfield in all portions of the cabin of the vehicle, the adaptive audio system may modify (or, in other words, adapt) audio playback to mute reproduction of the soundfield in one or more portions of the cabin of the vehicle (or, in other words, the vehicle cabin) based on status updates provided by the OMS. Muting the volume of playback in specific portions of the vehicle cabin may maintain, facilitate, or support continuation of the status of occupants (e.g., sleeping children) monitored by the OMS. By facilitating continuation of the status of the occupants, other passengers of the vehicle may continue to enjoy content (e.g., playback of the audio data) without distractions or otherwise interfering with the status of other occupants within the vehicle cabin, thereby improving enjoyment by occupants of the content reproduction provided by the infotainment system itself.

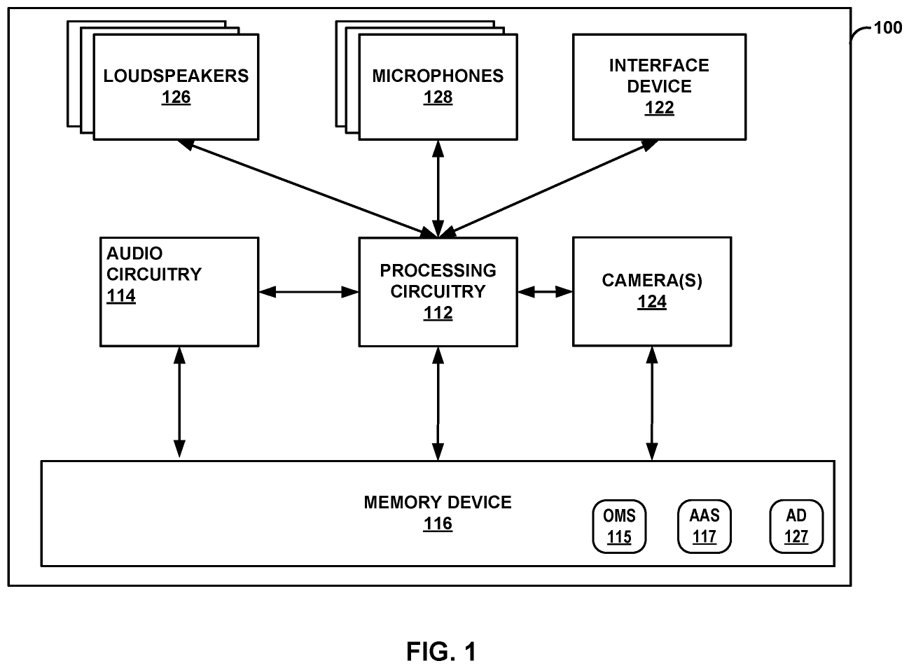

FIG. 1 is a block diagram illustrating an example vehicle configured to perform various aspects of the transparent audio mode techniques described in this disclosure. Vehicle 100 is assumed in the description below to be an automobile. However, the techniques described in this disclosure may apply to any type of vehicle capable of conveying occupant(s) in a cabin, such as a bus, a recreational vehicle (RV), a semi-trailer truck, a tractor or other type of farm equipment, a train car, a plane, a personal transport vehicle, and the like.

In the example of FIG. 1, the vehicle 100 includes processing circuitry 112, audio circuitry 114, and a memory device 116. In some examples, the processing circuitry 112 and the audio circuitry 114 may be formed as an integrated circuit (IC). For example, the IC may be considered as a processing chip within a chip package, and may be a system-on-chip (SoC).

Examples of the processing circuitry 112 and the audio circuitry 114 include, but are not limited to, one or more digital signal processors (DSPs), general purpose microprocessors, application specific integrated circuits (ASICs), field programmable logic arrays (FPGAs), fixed function circuitry, programmable processing circuitry, any combination of fixed function and programmable processing circuitry, or other equivalent integrated circuitry or discrete logic circuitry. The processing circuitry 112 may be the central processing unit (CPU) of the vehicle 100. In some examples, the audio circuitry 114 may be specialized hardware that includes integrated and/or discrete logic circuitry that provides the audio circuitry 114 with parallel processing capabilities.

The processing circuitry 112 may execute various types of applications, such as various occupant experience related applications including climate control interfacing applications, entertainment and/or infotainment applications, cellular phone interfaces (e.g., as implemented using Bluetooth® links), navigating applications, vehicle functionality interfacing applications, web or directory browsers, or other applications that enhance the occupant experience within the confines of the vehicle 100. The memory device 16 may store instructions for execution of the one or more applications.

The memory device 116 may include, be, or be part of the total memory for the vehicle 100. The memory device 116 may comprise one or more computer-readable storage media. Examples of the memory device 116 include, but are not limited to, a random access memory (RAM), an electrically erasable programmable read-only memory (EEPROM), flash memory, or other medium that can be used to carry or store desired program code in the form of instructions and/or data structures and that can be accessed by a computer or one or more processors (e.g., the processing circuitry 112 and/or the audio circuitry 114).

In some aspects, the memory device 116 may include instructions that cause the processing circuitry 112 and/or the audio circuitry 114 to perform the functions ascribed in this disclosure to the processing circuitry 112 and/or the audio circuitry 114. Accordingly, the memory device 16 may be a computer-readable storage medium (including a non-transitory computer-readable storage medium) having instructions stored thereon that, when executed, cause one or more processors (e.g., the processing circuitry 112 and/or the audio circuitry 114) to perform various functions attributed to the processing circuitry 112 and/or the audio circuitry 114.

The memory device 116 is a non-transitory storage medium. The term “non-transitory” indicates that the storage medium is not embodied in a carrier wave or a propagated signal. However, the term “non-transitory” should not be interpreted to mean that the memory device 116 is non-movable or that its contents are static. As one example, the memory device 116 may be removed from the vehicle 100, and moved to another device. As another example, memory, substantially similar to the memory device 116, may be inserted into one or more receiving ports of the vehicle 100. In certain examples, a non-transitory storage medium may store data that can, over time, change (e.g., in RAM).

As further shown in the example of FIG. 1, the vehicle 100 may include an interface device 122, camera(s) 124, multiple microphones 128, and one or more loudspeakers 126. In some examples, the interface device 122 may include one or more microphones that are configured to capture audio data within the vehicle 100. In some examples, the interface device 122 may include an interactive input/output display device, such as a touchscreen or other presence sensitive display. For instance, display devices that can form a portion of the interface device 122 may represent any type of passive screen on which images can be projected, or an active screen capable of projecting images (such as a light emitting diode (LED) display, an organic LED (OLED) display, liquid crystal display (LCD), or any other type of active display), with input-receiving capabilities built in.

Although shown as a single device in FIG. 1 for ease of illustration, the interface device 122 may include multiple user-facing devices that are configured to receive input and/or provide output. In various examples, the interface device 122 may include displays in wired or wireless communication with the vehicle 100, such as a heads-up display, a head-mounted display, an augmented reality computing device (such as “smart glasses”), a virtual reality computing device or display, a laptop computer or netbook, a mobile phone (including a so-called “smartphone”), a tablet computer, a gaming system, or another type of computing device capable of acting as an extension of or in place of a display integrated into the vehicle 100.

The interface device 122 may represent any type of physical or virtual interface with which a user may interface to control various functionalities of the vehicle 100. The interface device 122 may include physical buttons, knobs, sliders or other physical control implements. The interface device 122 may also include a virtual interface whereby an occupant of the vehicle 100 interacts with virtual buttons, knobs, sliders or other virtual interface elements via, as one example, a touch-sensitive screen. Occupant(s) may interface with the interface device 122 to control one or more of a climate within the vehicle 100, audio playback by the vehicle 100, video playback by the vehicle 100, transmissions (such as cell phone calls) through the vehicle 100, or any other operation capable of being performed by the vehicle 100.

The interface device 122 may also represent interfaces extended from the vehicle 100 when acting as an extension of or in place of a display integrated into the vehicle 100. That is, the interface device 122 may include virtual interfaces presented via the above noted HUD, augmented reality computing device, virtual reality computing device or display, tablet computer, or any other of the different types of extended displays listed above. The vehicle 100 may include a steering wheel for controlling a direction of travel of the vehicle 100, one or more pedals for controlling a rate of travel of the vehicle 100, one or more hand brakes, etc. In some examples, the steering wheel and pedals may be included in a particular in-cabin vehicle zone of the vehicle 100, such as in the driver zone or pilot zone.

For purposes of illustration, the processing circuitry 112, the audio circuitry 114, and the interface device 122 may form or otherwise support operation of a so-called head unit (which may also be referred to as a vehicle head unit). As such, reference to a head unit may refer to a computing device integrated within the vehicle 100 that includes the processing circuitry 112, the audio circuitry 114, and the interface device 122. The processing circuitry 112 may execute an operating system (OS) having a kernel (which is an OS layer that facilitates interactions with underlying hardware of the head unit and other connected hardware components, and executes in protected OS space) that supports execution of applications in an application space provided by the OS.

The camera(s) 124 of the vehicle 100 may represent one or more image and/or video capture devices configured to capture image data (where a sequence of image data may form video data). The vehicle 100 may include a single camera capable of capturing 360 degrees of image/video data, or multiple cameras configured to capture a portion of the surroundings of the vehicle 100 (where each portion may be stitched together to form 360 degrees of image/video data). In some examples, the cameras 124 may only capture discrete portions of (and not all portions necessary to form) 360 degrees of image/video data. In other examples, the cameras 124 may enable capture of a three-dimensional image/video data representative of an entire visual scene surrounding the vehicle 100.

The cameras 124 may be disposed in a single location on a body of the vehicle 100 (e.g., a roof of the vehicle 100) or multiple locations around the body of and externally directed from the vehicle 100 to capture image/video data representative of an external visual scene in which the vehicle 100 operates. The cameras 124 may assist in various levels of autonomous driving, safety systems (e.g., lane assist, dynamic cruise control, etc.), vehicle operation (e.g., backup cameras for assisting in backing up the vehicle 100), and the like.

The cameras 124 may also be disposed within a cabin of the vehicle 100. The cameras 124 may capture images depicting the interior of the cabin of the vehicle 100 so as to assess a state of occupants within the vehicle 100. For example, the cameras 124 may capture images of the operator of the vehicle 100 (or, in other words, a driver) to assess awareness of the operational state in which the vehicle 100 is operating. The cameras 124 may also identify one or more occupants that are passengers of the vehicle 100 and identify various states of the passengers (e.g., sleeping, consuming media, talking, resting, etc.).

The microphones 128 of the vehicle 100 may represent a microphone array representative of a number of different microphones 128 placed external to the vehicle 100 in order to capture a sound scene of an environment within which the vehicle 100 is operating. The microphones 128 may each represent a transducer that converts sound waves into electrical signals (which may be referred to as audio signals, and when processed into digital signals, audio data). One or more of the microphones 128 may represent reference microphones and/or error microphones for performing audio signal processing (e.g., wind noise cancellation, active noise cancellation, etc.).

The microphones 128 may also be disposed within the cabin of the vehicle 100. As noted above, the microphones 128 may include reference microphones and/or error microphones for performing audio signal processing (e.g., active noise cancellation, etc.). The microphones 128 may be disposed internally within the cabin of the vehicle 100 at particular zones (e.g., an operator—or in other words, driver—zone, a front passenger zone, a rear passenger zone (including both driver-side rear passenger zone and passenger-side rear passenger zone), etc. The microphones 128 may capture audio data representative of a soundfield in a respective driver/passenger/rear-passenger zone.

The loudspeakers 126 represent components of the vehicle 100 that reproduce a soundfield based on audio signals provided directly or indirectly by the processing circuitry 112 and/or the audio circuitry 114. For instance, the loudspeakers 126 may generate pressure waves based on one or more electrical signals received from the processing circuitry 112 and/or the audio circuitry 114. The loudspeakers 126 may include various types of speaker hardware, including full-range driver-based loudspeakers, individual loudspeakers that include multiple range-specific dynamic drivers, or loudspeakers that include a single dynamic driver such as a tweeter or a woofer.

The audio circuitry 114 may be configured to perform audio processing with respect to audio signals/audio data captured via the microphones 128 in order to drive the loudspeakers 126. The audio circuitry 114 may also receive audio signals/audio data from the processing circuitry 112 that the audio circuitry 114 may process in order to drive the loudspeakers 126. The term “drive” as used herein may refer to a process of providing audio signals to the loudspeakers 126, which includes a driver by which to convert the audio signals into pressure waves (which is another way of referring to sound waves). The term “drive” refers to providing such audio signals to the driver of the loudspeakers 126 in order to reproduce a soundfield (which is another way of referring to a sound scene) represented by the audio signals.

Many vehicles, such as the vehicle 100, are equipped with entertainment or infotainment systems (which is another way to refer to a vehicle headunit), which reproduce a soundfield, based on audio data (or in other words, audio signals), via loudspeakers, such as the loudspeakers 126. In addition, many vehicles, such as the vehicle 100, include an occupant monitoring system (OMS) 115, which is software executed by, in this example, the processing circuitry 112, the audio circuitry 114, etc. and that interacts with the interface devices 122, the cameras 124, the microphones 128, etc. That is, the OMS 115 may monitor a status of occupants within the cabin of the vehicle 100.

The infotainment system may be configured to adapt operating conditions (e.g., present safety notifications, such as the status of the seatbelt for a passenger) dependent upon status updates provided by the OMS 115. That is, the infotainment systems may use status updates provided by the OMS 115 to adapt user interfaces, activate one or more of the cameras 124, activate one or more of the microphones 128, etc. to enable occupant aware functionality regarding safety, awareness, activity, etc. occurring within the cabin of the vehicle 100. However, the infotainment systems may not adapt reproduction of a soundfield based on OMS status updates concerning the various occupants within the vehicle 100.

In accordance with various aspects of the techniques described in this disclosure, a vehicle 100 or other device (e.g., a vehicle head unit) may implement an adaptive audio system (AAS) 117, interfacing with the OMS 115 to receive status updates regarding occupants residing within a cabin of the vehicle 100. The adaptive audio system 117 may receive status updates indicating that, as an example, a rear passenger residing within a rear passenger zone of the cabin of the vehicle 100 is sleeping (e.g., a child—baby—is sleeping). Rather than fully reproduce the soundfield throughout the vehicle 100, the adaptive audio system 117 may modify playback of the audio data (AD) 127 based on the status updated provided by the OMS 115 (where, in this example, the adaptive audio system 117 may mute the playback of the audio data 127 in the rear-passenger zone of the cabin of the vehicle 100).

In operation, the AAS 117 may obtain, from the OMS 115, a state of an occupant residing within a cabin of the vehicle 100. The OMS 115 (executed by processing circuitry 112) may invoke one or more of the cameras 124 to capture occupant visual data (not shown in the example of FIG. 1 for ease of illustration purposes) that may include images, videos, etc. of occupants residing within the vehicle cabin. The OMS 115 may also invoke one or more of the microphones 128 to further capture occupant audio data (which may be similar to the AD 127) in one or more zones of the vehicle cabin.

The OMS 115 may determine, based on the occupant visual data and the occupant audio data, a current status of each occupant residing within the cabin of the vehicle 100. The OMS 115 may represent one or more trained machine learning modules that are applied to the occupant visual data and/or the occupant audio data to identify a state of each occupant residing within the vehicle cabin (which again may be another way to refer to the cabin of the vehicle 100). The OMS 115 may identify a current state of each occupant residing within the vehicle cabin, where such states include awareness levels (such as observant, awake, active, distracted, asleep, resting, etc.) based on the occupant visual data and/or the occupant audio data.

The OMS 115 may push status updates to the AAS 117, where “push” refers to an application programming interface (API) in which a corresponding application (i.e., the AAS 117 in this example) registers for occupant status updates and receives various software notifications (e.g., exceptions or interrupts) that signal a new occupant status is available for processing. Alternatively, or in conjunction, the OMS 115 may operate in a pull status updates for the AAS 117 in which the AAS 117 issues requests by which to obtain the occupant status updates responsive to indication from the OMS 115 that the occupant status for a particular one of the occupants residing within the vehicle cabin has changed.

Regardless, the AAS 117 (as executed by the processing circuitry and/or the audio circuitry 114) may obtain the occupant status update defining the state of the occupant residing within the cabin of the vehicle 100. The AAS 117 may next modify, based on the state of the occupant residing within the cabin of the vehicle 100, playback of the audio data 127 within at least a portion of the cabin of the vehicle 100 to obtain modified playback data for the audio data 127.

For example, the OMS 115 may determine that a rear passenger in the cabin of the vehicle 100 is asleep (e.g., a child is sleeping in the driver-side rear passenger zone of the cabin of the vehicle). The OMS 115 may, in this example, interface with the AAS 117 (via an API exposed by the AAS 117) to pass the state update indicating that the passenger in the driver-side rear passenger zone of the cabin of the vehicle 100 is sleeping. The AAS 117 may, responsive to being invoked and passing the state update, is configured to generate modified audio playback data indicating that audio playback of the AD 127 is to be muted in the driver-side rear passenger zone of the cabin of the vehicle 100.

The AAS 117 may then, based on the modified audio playback data, render the AD 127 such that a gain associated with a channel of the AD 127 associated with the driver-side rear passenger zone of the vehicle cabin is muted. In this respect, the AAS 117 may mute, responsive to the sleeping state indicating that the occupant residing within the (in this instance, driver-side) rear passenger zone of the cabin of the vehicle 100 is sleeping, audio playback in the (in this instance, driver-side) rear passenger zone of the cabin of the vehicle 100. The AAS 117 may then output the rendered channels to the loudspeakers 127 in order to reproduce, based on the modified playback data, the soundfield (represented by the AD 127).

In this respect, various aspects of the techniques may improve operation of the infotainment system (which is another way to refer to a vehicle head unit) itself. For example, rather than indiscriminately reproduce the soundfield represented by the AD 127 in all portions of the cabin of the vehicle 100, the AAS 117 may modify (or, in other words, adapt) audio playback to mute reproduction of the soundfield in one or more portions of the cabin of the vehicle 100 (or, in other words, the vehicle cabin) based on status updates provided by the OMS 115. Muting the volume of playback in specific portions of the vehicle cabin may maintain, facilitate, or support continuation of the status of occupants (e.g., sleeping children) monitored by the OMS 115. By facilitating continuation of the status of the occupants, other passengers of the vehicle 100 may continue to enjoy content (e.g., playback of the audio data) without distractions or otherwise interfering with the status of other occupants within the vehicle cabin, thereby improving enjoyment by occupants of the content reproduction provided by the infotainment system itself.

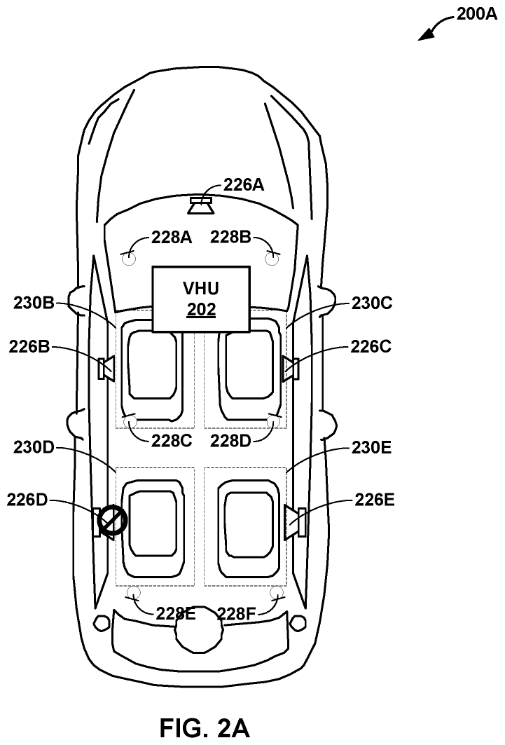

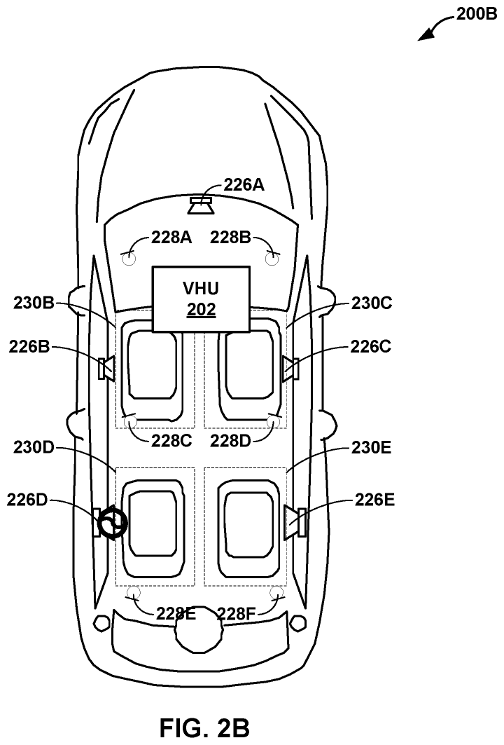

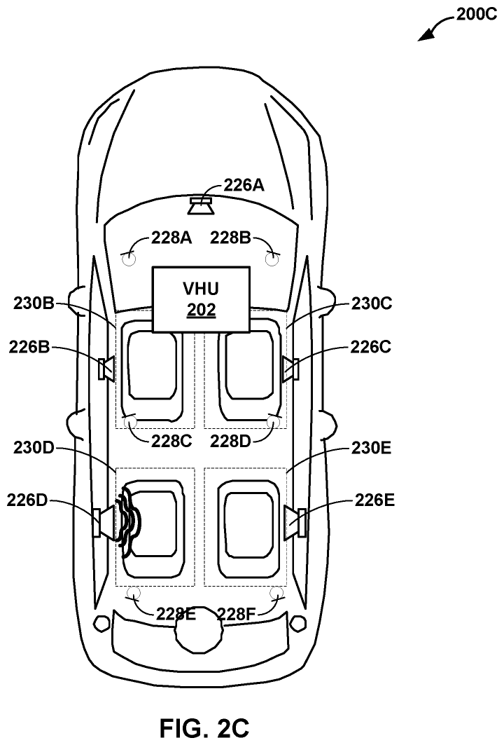

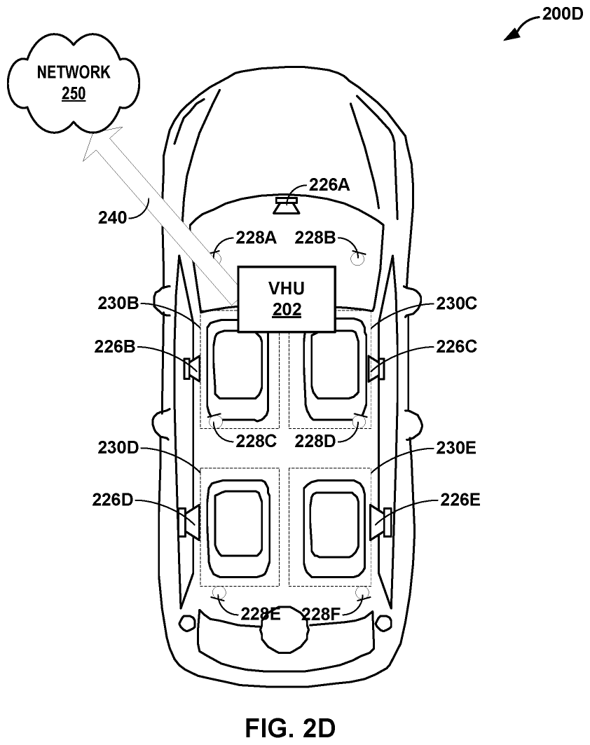

FIGS. 2A-2D are diagrams illustrating example operation of the AAS shown in the example of FIG. 1 in perform various aspects of the adaptive audio playback techniques described in this disclosure. Referring first to the example of FIG. 2A, a vehicle 200A is shown that that includes a vehicle head unit (VHU) 202 (“VHU 202”) that represents an example of the processing circuitry 112, the audio circuitry 114, and/or interface devices 122, etc. shown in the example of FIG. 1. VHU 202 may represent one example of a device configured to perform (e.g., execute instructions represented by the OMS 115 and/or the AAS 117 that cause the processing circuitry 112 and/or the audio circuitry 114-112/114) various aspect of the adaptive audio techniques described in this disclosure.

As shown in the example of FIG. 2A, the VHU 202 may interface with loudspeakers 226A-226E (“loudspeakers 226”), which may represent examples of the loudspeakers 126 shown in the example of FIG. 1. The VHU 202 may also interface with microphones 228A-228F (“microphones 228”), which again may represent examples of the microphones 128. While described with respect to five (5) loudspeakers, i.e., the loudspeakers 226, and the six (6) microphones 228, the VHU 202 may interface with more or less loudspeakers 226 and more or less microphones 228.

In any event, the VHU 202 may execute the OMS 115 (which in other examples may be executed by other processing circuitry disposed within other locations within the vehicle 200A—including different devices unassociated with the vehicle 200A—such as smartphones, smartwatches, smart glasses, tablet computers, laptop computers, gaming systems, portable computing devices, etc.). In any event, the OMS 115 may interface with the cameras 124 (which are not shown in the example of FIG. 2A for ease of illustration purposes) to obtain occupant visual data representative of the interior (or, in other words, the cabin) of the vehicle 200A. The OMS 115 may also interface with microphones 228 to obtain occupant audio data representative of the interior of the vehicle 200A.

In the example of FIG. 2A, the vehicle 200A includes a cabin (or, in other words, an interior of the vehicle 200A) divided into separate zones 230B-230E (“zones 230”), where the zone 230B represents a driver-side front passenger (or, in other words, occupant) zone, the zone 230C represents a passenger side front passenger zone, the zone 230D represents a driver-side rear passenger zone, and the zone 230E represents a passenger-side rear passenger zone. The OMS 115 may interface with the cameras 124 and/or the microphone 228A to capture the occupant visual data and/or occupant audio data to identify whether a driver (or, in other words, operator) of the vehicle 200A is present. When the driver/operator is present, the OMS 115 may then interface with the AAS 117 to provide a status of the driver/operator of the vehicle 200A.

Likewise, the OMS 115 may interface with various the cameras 124 and/or the microphones 228 used for monitoring each of the zones 230 in order to determine a presence of an occupant at each of the zones 230. The OMS 115 may next, when an occupant is present in any of the zones 230, analyze the visual data and/or the audio data to determine a current status of the occupant(s) residing at each of the zones 230 of the vehicle 200A.

That is, in addition to interfacing with the cameras 124 and the microphone 228A for the driver-side front zone 230B, the OMS 115 may interface with the cameras 124 and the microphone 228B to obtain the occupant visual data and/or the occupant audio data for the front passenger-side zone 230C. The OMS 115 may interface with the cameras 124 and/or the microphone 228C and/or 228E to obtain the occupant visual data and/or the occupant audio data for the driver-side rear passenger zone 230D. The OMS 115 may interface with the cameras 124 and the microphone 228C and/or 228F to obtain the occupant visual data and/or the occupant audio data for the passenger-side rear passenger zone 230E.

In the example of FIG. 2A, the OMS 115 may determine that an occupant is present at the driver-side rear passenger zone 230D based on the occupant visual data and/or the occupant audio data captured for the driver-side rear passenger zone 230D. The OMS 115 may analyze the occupant visual data and/or the occupant audio data to determine a current status of the occupant residing in the driver-side rear passenger zone 230D. The OMS 115 may, in this example, determine that the occupant residing in the driver-side rear passenger zone 230D, is sleeping (or, in other words, asleep). The OMS 115 may interface (via the above noted API) with the AAS 117 to pass the updated status of the occupant residing in the driver-side rear passenger zone 230D.

Responsive to receiving the updated status of the occupant residing in the driver-side rear passenger zone 230D, the AAS 117 may reduce a gain (or in other words mute) associated with a speaker channel rendered for the speaker 226D located proximate (or, in other words, closest) to the driver-side rear passenger zone 230D. The AAS 117 may mute the speaker channel rendered for the speaker 226D using modified playback data that indicates a channel-specific volume (e.g., a reduced volume or no volume) for the particular loudspeaker, i.e., the loudspeaker 226D in this example.

The AAS 117 may then render, based on the modified playback data, the AD 127 to obtain one or more speaker feeds, which represent electrical signals for driving the loudspeakers 226. The AAS 117 may output the speaker feeds to the loudspeakers 226, which may reproduce the soundfield based on the speaker feeds. As shown in the example of FIG. 2A, the loudspeaker 226D does not reproduce any soundfield as denoted by the circle with the straight line strikethrough.

Referring next to the example of FIG. 2B, a vehicle 200B may represent another example of the vehicle 100 shown in the example of FIG. 1. The vehicle 200B may be similar, if not substantially similar, to the vehicle 200B, except the AAS 117 may interface with the various microphones 228 to identify a reference audio signal and/or an error audio signal that may be used for noise cancellation (e.g., active noise cancellation).

Responsive to receiving the updated status of the occupant residing in the driver-side rear passenger zone 230D, the AAS 117 may perform, responsive to the sleeping state indicating that the occupant residing within the rear passenger zone (e.g., the zone 230D) of the cabin of the vehicle is sleeping, active noise cancellation with respect to the driver-side rear passenger zone 230D of the cabin of the vehicle 200B that includes modifying the audio data prior to playback within the driver-side rear passenger zone 230D to limit the soundfield from being reproduced in the driver-side rear passenger zone 230D.

Active noise cancellation may involve generating a counter wave that accounts for (meaning a wave that is 180 degrees out of phase with) audio soundfields captured by the reference and/or error microphones (which may be represented as one or more of the microphones 128/228). As soundfields exist in a physical domain, counter sound waves may cancel soundwaves output by other ones of the loudspeakers 226 that may reduce or eliminate unwanted noise (including the reproduced soundfield based on the AD 127) in any one of the zones 230. Active noise cancellation is denoted in the example of FIG. 2B as a circle with wavey strikethrough lines.

In the example of FIG. 2C, a vehicle 200C may represent another example of the vehicle 100 shown in the example of FIG. 1. The vehicle 200C may be similar, if not substantially similar, to the vehicle 200A and/or 200B, except the AAS 117 may as an alternative, or in addition to the examples described herein with respect to the examples of FIG. 2A-2D, both mute and/or perform anti-noise cancellation with respect to the speaker channel for the loudspeakers 226D as well as replace, responsive to the sleeping state indicating that the occupant residing within the rear passenger zone 230D of the cabin of the vehicle 200C is sleeping, portion of the audio data 127 with soothing audio data to facilitate the sleeping state of the occupant residing within the rear passenger zone 230D of the cabin of the vehicle 300C.

The VHU 202 executing the AAS 117 may retrieve the soothing audio data from a streaming audio source, from a dedicated on-board storage for the soothing audio data, and/or from microphones 228 (e.g., a recording of another occupant residing within the vehicle 200C—or from microphones of devices associated with the vehicle 200C, such as a smartphone, smartwatch, smart glasses, laptop computer, tablet computer, etc. of a passenger associated with the vehicle 200C). AAS 117 may retrieve or otherwise obtain the soothing audio data from most any source, including sources configured via the VHA 202 for sourcing the soothing audio data. The reproduction of the soundfield based on the soothing audio data is shown as bent soundfield lines in the example of FIG. 2C.

In the example of FIG. 2D, a vehicle 200D may represent another example of the vehicle 100 shown in the example of FIG. 1. The vehicle 200D may be similar, if not substantially similar, to the vehicle 200A-200C, except the VHU 2-2 may as an alternative, or in addition to the examples described herein with respect to the examples of FIG. 2A-2D, alert, responsive to detect the state of the occupant is the zones 230, alert the operator of the vehicle that the occupant in the rear child zone of the cabin of the vehicle 200D is a child. In some instances, the VHU 202 may adjust, responsive to detecting the state of the occupant in the rear passenger zone (i.e., the rear passenger zones 230D and/or 230E in this example), a heating, ventilation, and air conditioning (HVAC) setting for the rear passenger zone (i.e., the rear passenger zones 230D and/or 230E in this example) of the cabin of the vehicle 200D.

In addition, the VHU 202 may determine an operational state of the vehicle 200D, which may refer to a state of the vehicle 200D such as driving, waiting, stalled, parked, temperature inside, temperature outside, etc. The VHU 202 may perform, based on the state of the occupant residing within the vehicle 200D and the operational state of the vehicle 200D, a safety action to facilitate safety with respect to the occupant residing within the vehicle.

That is, an operator of the vehicle 200D may leave the vehicle 200D with another occupant present inside the cabin of the vehicle 200D. The interior of the vehicle 200D may exceed safe temperatures (both above or below habitable temperatures—e.g., 50 degrees Fahrenheit through 80 degrees Fahrenheit) that dictate habitable conditions when the vehicle 200D is parked without access to HVAC systems (e.g., when parked and locked). In some examples, the VHU 202 may automatically turn on HVAC systems, when an occupant of the vehicle 200D is detected and when exterior (or, in other words, external) conditions exceed habitable conditions), to adjust an internal temperature of the vehicle 200D to maintain habitable conditions within the vehicle 200D. Automatically adjusting (meaning adjusting without input or other interactions with the operator/owner/driver of the vehicle 200D) the temperature provided by the HVAC systems of the vehicle 200D to maintain habitable conditions may represent one example of the safety action.

In other examples, the VHU 202 may perform the safety action by way of initiating, responsive to the operational state of the vehicle 200D indicating that the occupant is still residing within the vehicle 200D and the state of the occupant indicating that the occupant is still residing within the vehicle 200D (even when locked), a phone call (e.g., via a cellular network using cellular phone services) to one or more of an owner of the vehicle 200D, an (possibly temporary) operator of the vehicle 200D, a preferred contact for the vehicle 200D (possibly as specified via settings exposed via an operating system—OS—executed by the VHU 202), and emergency services (e.g., 911 services in the United States).

While described with respect to a cellular phone call, the VHU 202 may initiate a text message in addition to or as an alternative to the cellular phone call, where such cellular services may utilize one or more cellular services including data cellular services. In the example of FIG. 2D, the VHU 202 may initiate a cellular safety service 240 (which may represent a cellular phone call, cellular text messages, cellular data messaging, etc.) with a network 250, which may represent a wireless network connection via a cellular standard or other wireless standard (such as WiFi™) with a public network (such as the Internet) and/or a private network.

In addition, or as an alternative, to the above safety operations, the VHU 202 may initiate, responsive to the operational state of the vehicle 200D indicating that the occupant is still residing within the vehicle 200D and the state of the occupant indicating that the occupant is still residing within the vehicle 200D, a safety alarm to alert people nearby of the occupant within the vehicle 200D. This safety alarm may include honking the horn of the vehicle 200D, triggering an alarm via a network (e.g., via an application monitoring nearby cars and connected to a wireless network of the vehicle 200D), flashing headlights of the vehicle 200D, revving the engine, etc.

In some instances, the VHU 202 may perform safety actions according to a configurable escalation policy. The escalation policy may provide a prioritized action list in which one or more of the above safety action may be performed according to a time-based, temperature-based (e.g., internal temperature, external temperature, or some combination of both the internal temperature and the external temperature), action-based (e.g., with respect to the vehicle 200D, such as attempting to open and/or unlock a door of the vehicle 200D) or other criteria-based metric.

For example, a child may reside in the driver-side rear passenger zone 230D while the vehicle 200D is locked, not operational, and parked, the VHU 202 may perform, according the configurable escalation policy, a safety action including initiating a cellular phone call. The VHU 202 may, when determining that the cellular phone call went unanswered, continue to escalate the safety action according to the escalation policy by issuing a text message requesting a response. The VHU 20s may, when determining that the text message went unanswered, continue to escalate the safety action according to the escalation policy by issuing the safety alarm. The owner or other authorized operator of the vehicle 200D may configure the escalation policy in terms of which mode of communication (meaning, for example, cellular phone call, text message, safety alarm, etc.) is preferred and the order in which each mode of communication is to be performed.

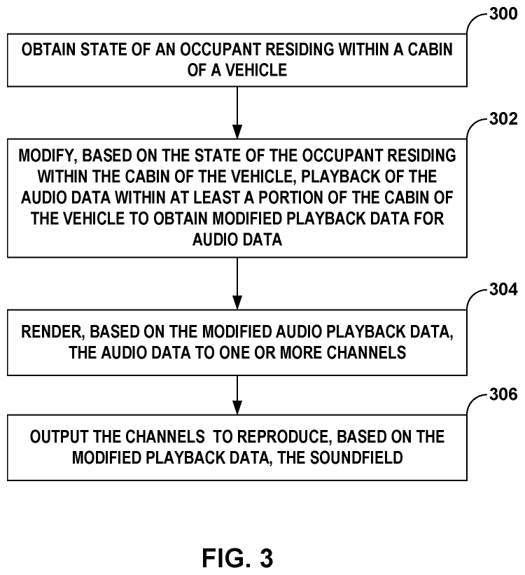

FIG. 3 is a flowchart illustrating example operation of the vehicle shown in the example of FIG. 1 in performing various aspects of the adaptive audio playback techniques described in this disclosure. the AAS 117 may obtain, from the OMS 115, a state of an occupant residing within a cabin of the vehicle 100 (300). The OMS 115 (executed by processing circuitry 112) may invoke one or more of the cameras 124 to capture occupant visual data (not shown in the example of FIG. 1 for ease of illustration purposes) that may include images, videos, etc. of occupants residing within the vehicle cabin. The OMS 115 may also invoke one or more of the microphones 128 to further capture occupant audio data (which may be similar to the AD 127) in one or more zones of the vehicle cabin.

The AAS 117 may next modify, based on the state of the occupant residing within the cabin of the vehicle 100, playback of the audio data 127 within at least a portion of the cabin of the vehicle 100 to obtain modified playback data for the audio data 127 (302). For example, the OMS 115 may determine that a rear passenger in the cabin of the vehicle 100 is asleep (e.g., a child is sleeping in the driver-side rear passenger zone of the cabin of the vehicle). The OMS 115 may, in this example, interface with the AAS 117 (via an API exposed by the AAS 117) to pass the state update indicating that the passenger in the driver-side rear passenger zone of the cabin of the vehicle 100 is sleeping. The AAS 117 may, responsive to being invoked and passing the state update, is configured to generate modified audio playback data indicating that audio playback of the AD 127 is to be muted in the driver-side rear passenger zone of the cabin of the vehicle 100.

The AAS 117 may then, based on the modified audio playback data, render the AD 127 (304) such that, in this example, a gain associated with a channel of the AD 127 associated with the driver-side rear passenger zone of the vehicle cabin is muted. In this respect, the AAS 117 may mute, responsive to the sleeping state indicating that the occupant residing within the (in this instance, driver-side) rear passenger zone of the cabin of the vehicle 100 is sleeping, audio playback in the (in this instance, driver-side) rear passenger zone of the cabin of the vehicle 100. The AAS 117 may then output the rendered channels to the loudspeakers 127 in order to reproduce, based on the modified playback data, the soundfield (represented by the AD 127) (306).

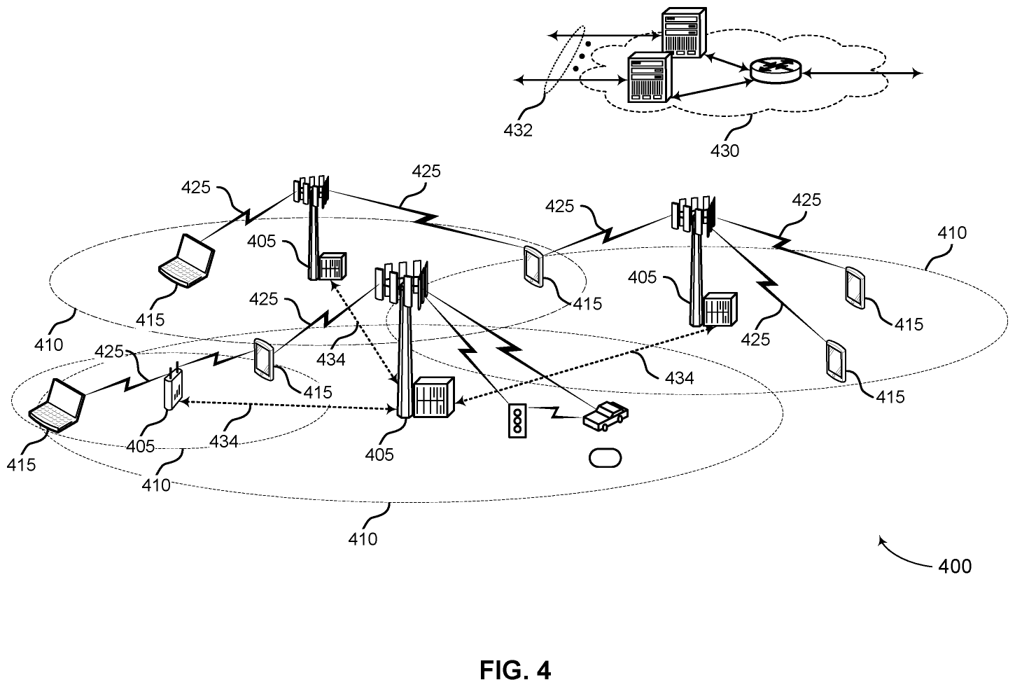

FIG. 4 illustrates an example of a wireless communications system 400 in accordance with aspects of the present disclosure. The wireless communications system 400 includes base stations 405, UEs 415, and a core network 430. In some examples, the wireless communications system 400 may be a Long Term Evolution (LTE) network, an LTE-Advanced (LTE-A) network, an LTE-A Pro network, a 5^{th} generation cellular network, or a New Radio (NR) network. In some cases, wireless communications system 100 may support enhanced broadband communications, ultra-reliable (e.g., mission critical) communications, low latency communications, or communications with low-cost and low-complexity devices. The wireless communication system 400 may represent one example of the network 250 shown in the example of FIG. 2D.

Base stations 405 may wirelessly communicate with UEs 415 via one or more base station antennas. Base stations 405 described herein may include or may be referred to by those skilled in the art as a base transceiver station, a radio base station, an access point, a radio transceiver, a NodeB, an eNodeB (eNB), a next-generation NodeB or giga-NodeB (either of which may be referred to as a gNB), a Home NodeB, a Home eNodeB, or some other suitable terminology. Wireless communications system 400 may include base stations 105 of different types (e.g., macro or small cell base stations). The UEs 415 described herein may be able to communicate with various types of base stations 405 and network equipment including macro eNBs, small cell eNBs, gNBs, relay base stations, and the like.

Each base station 405 may be associated with a particular geographic coverage area 410 in which communications with various UEs 415 are supported. Each base station 405 may provide communication coverage for a respective geographic coverage area 410 via communication links 425, and communication links 425 between a base station 405 and a UE 415 may utilize one or more carriers. Communication links 425 shown in wireless communications system 400 may include uplink transmissions from a UE 415 to a base station 405, or downlink transmissions from a base station 405 to a UE 415. Downlink transmissions may also be called forward link transmissions while uplink transmissions may also be called reverse link transmissions.

The geographic coverage area 410 for a base station 405 may be divided into sectors making up a portion of the geographic coverage area 410, and each sector may be associated with a cell. For example, each base station 405 may provide communication coverage for a macro cell, a small cell, a hot spot, or other types of cells, or various combinations thereof. In some examples, a base station 405 may be movable and therefore provide communication coverage for a moving geographic coverage area 410. In some examples, different geographic coverage areas 410 associated with different technologies may overlap, and overlapping geographic coverage areas 410 associated with different technologies may be supported by the same base station 405 or by different base stations 405. The wireless communications system 400 may include, for example, a heterogeneous LTE/LTE-A/LTE-A Pro, 5^{th} generation, or NR network in which different types of base stations 405 provide coverage for various geographic coverage areas 410.

UEs 415 may be dispersed throughout the wireless communications system 400, and each UE 415 may be stationary or mobile. A UE 415 may also be referred to as a mobile device, a wireless device, a remote device, a handheld device, or a subscriber device, or some other suitable terminology, where the “device” may also be referred to as a unit, a station, a terminal, or a client. A UE 415 may also be a personal electronic device such as a cellular phone, a personal digital assistant (PDA), a tablet computer, a laptop computer, or a personal computer. In examples of this disclosure, a UE 415 may be any of the audio sources described in this disclosure, including a VR headset, an XR headset, an AR headset, a vehicle, a smartphone, a microphone, an array of microphones, or any other device including a microphone or is able to transmit a captured and/or synthesized audio stream. In some examples, a synthesized audio stream may be an audio stream that that was stored in memory or was previously created or synthesized. In some examples, a UE 415 may also refer to a wireless local loop (WLL) station, an Internet of Things (IoT) device, an Internet of Everything (IoE) device, or a machine-type communication (MTC) device, or the like, which may be implemented in various articles such as appliances, vehicles, meters, or the like.

Some UEs 415, such as MTC or IoT devices, may be low cost or low complexity devices, and may provide for automated communication between machines (e.g., via Machine-to-Machine (M2M) communication). M2M communication or MTC may refer to data communication technologies that allow devices to communicate with one another or a base station 405 without human intervention. In some examples, M2M communication or MTC may include communications from devices that exchange and/or use audio metadata that may include timing metadata used to affect audio streams and/or audio sources.

In some cases, a UE 415 may also be able to communicate directly with other UEs 415 (e.g., using a peer-to-peer (P2P) or device-to-device (D2D) protocol). One or more of a group of UEs 415 utilizing D2D communications may be within the geographic coverage area 410 of a base station 405. Other UEs 415 in such a group may be outside the geographic coverage area 410 of a base station 405, or be otherwise unable to receive transmissions from a base station 405. In some cases, groups of UEs 415 communicating via D2D communications may utilize a one-to-many (1:M) system in which each UE 415 transmits to every other UE 415 in the group. In some cases, a base station 405 facilitates the scheduling of resources for D2D communications. In other cases, D2D communications are carried out between UEs 415 without the involvement of a base station 405.

Base stations 405 may communicate with the core network 430 and with one another. For example, base stations 405 may interface with the core network 430 through backhaul links 432 (e.g., via an S1, N2, N3, or other interface). Base stations 405 may communicate with one another over backhaul links 434 (e.g., via an X2, Xn, or other interface) either directly (e.g., directly between base stations 405) or indirectly (e.g., via core network 430).

In some cases, wireless communications system 400 may utilize both licensed and unlicensed radio frequency spectrum bands. For example, wireless communications system 400 may employ License Assisted Access (LAA), LTE-Unlicensed (LTE-U) radio access technology, or NR technology in an unlicensed band such as the 5 GHz Industrial, Scientific, Medical (ISM) band. When operating in unlicensed radio frequency spectrum bands, wireless devices such as base stations 405 and UEs 415 may employ listen-before-talk (LBT) procedures to ensure a frequency channel is clear before transmitting data. In some cases, operations in unlicensed bands may be based on a carrier aggregation configuration in conjunction with component carriers operating in a licensed band (e.g., LAA). Operations in unlicensed spectrum may include downlink transmissions, uplink transmissions, peer-to-peer transmissions, or a combination of these. Duplexing in unlicensed spectrum may be based on frequency division duplexing (FDD), time division duplexing (TDD), or a combination of both.

There are a number of different ways to represent a soundfield. Example formats include channel-based audio formats, object-based audio formats, and scene-based audio formats. Channel-based audio formats refer to the 5.1 surround sound format, 7.1 surround sound formats, 22.2 surround sound formats, or any other channel-based format that localizes audio channels to particular locations around the listener in order to recreate a soundfield.

Object-based audio formats may refer to formats in which audio objects, often encoded using pulse-code modulation (PCM) and referred to as PCM audio objects, are specified in order to represent the soundfield. Such audio objects may include location information, such as location metadata, identifying a location of the audio object relative to a listener or other point of reference in the soundfield, such that the audio object may be rendered to one or more speaker channels for playback in an effort to recreate the soundfield. The techniques described in this disclosure may apply to any of the following formats, including scene-based audio formats, channel-based audio formats, object-based audio formats, or any combination thereof.

Scene-based audio formats may include a hierarchical set of elements that define the soundfield in three dimensions. One example of a hierarchical set of elements is a set of spherical harmonic coefficients (SHC). The following expression demonstrates a description or representation of a soundfield using SHC:

$${p}_{i} ( t , {r}_{r} , {θ}_{r} , {φ}_{r} ) = {\sum }_{ω = 0}^{∞} [ 4 π {\sum }_{n = 0}^{∞} {j}_{n} ( {kr}_{r} ) {\sum }_{m = - n}^{n} {A}_{n}^{m} ( k ) {Y}_{n}^{m} ( {θ}_{r} , {φ}_{r} ) ] {e}^{j ω t} ,$$

The expression shows that the pressure pᵢ at any point {rᵣ, θᵣ, φᵣ} of the soundfield, at time t, can be represented uniquely by the SHC, Aₙ^{m}(k). Here,

$$k = \frac{ω}{c} ,$$

c is the speed of sound (˜343 m/s), {rᵣ, θᵣ, φᵣ,} is a point of reference (or observation point), jₙ(⋅) is the spherical Bessel function of order n, and Yₙ^{m}(θᵣ, φᵣ) are the spherical harmonic basis functions (which may also be referred to as a spherical basis function) of order n and suborder m. It can be recognized that the term in square brackets is a frequency-domain representation of the signal (e.g., S(ω, rᵣ, θᵣ, φᵣ)) which can be approximated by various time-frequency transformations, such as the discrete Fourier transform (DFT), the discrete cosine transform (DCT), or a wavelet transform. Other examples of hierarchical sets include sets of wavelet transform coefficients and other sets of coefficients of multiresolution basis functions.

The SHC Aₙ^{m}(k) can either be physically acquired (e.g., recorded) by various microphone array configurations or, alternatively, they can be derived from channel-based or object-based descriptions of the soundfield. The SHC (which also may be referred to as ambisonic coefficients) represent scene-based audio, where the SHC may be input to an audio encoder to obtain encoded SHC that may promote more efficient transmission or storage. For example, a fourth-order representation involving (1+4)² (25, and hence fourth order) coefficients may be used.

As noted above, the SHC may be derived from a microphone recording using a microphone array. Various examples of how SHC may be physically acquired from microphone arrays are described in Poletti, M., “Three-Dimensional Surround Sound Systems Based on Spherical Harmonics,” J. Audio Eng. Soc., Vol. 53, No. 11, 2005 November, pp. 1004-1025.

The following equation may illustrate how the SHCs may be derived from an object-based description. The coefficients Aₙ^{m}(k) for the soundfield corresponding to an individual audio object may be expressed as:

Aₙ^{m}(k)=g(ω)(−4πik)hₙ⁽²⁾(krₛ)Yₙ^{m′}(θₛ, φₛ),

where i is \sqrt{−1}, hₙ⁽²⁾(⋅) is the spherical Hankel function (of the second kind) of order n, and {rₛ, θₛ, φₛ} is the location of the object. Knowing the object source energy g(ω) as a function of frequency (e.g., using time-frequency analysis techniques, such as performing a fast Fourier transform on the pulse code modulated—PCM—stream) may enable conversion of each PCM object and the corresponding location into the SHC Aₙ^{m}(k). Further, it can be shown (since the above is a linear and orthogonal decomposition) that the Aₙ^{m}(k) coefficients for each object are additive. In this manner, a number of PCM objects can be represented by the Aₙ^{m}(k) coefficients (e.g., as a sum of the coefficient vectors for the individual objects). The coefficients may contain information about the soundfield (the pressure as a function of three dimensional (3D) coordinates), and the above represents the transformation from individual objects to a representation of the overall soundfield, in the vicinity of the observation point {rᵣ, θᵣ, φᵣ}.

According to the techniques of this disclosure, individual audio streams may be restricted from rendering or may be rendered on a temporary basis based on timing information, such as a time or a duration. Certain individual audio streams or clusters of audio streams may be enabled or disabled for a fixed duration for better audio interpolation. Accordingly, the techniques of this disclosure provide for a flexible manner of controlling access to audio streams based on time.

It should be noted that the methods described herein describe possible implementations, and that the operations and the steps may be rearranged or otherwise modified and that other implementations are possible. Further, aspects from two or more of the methods may be combined.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In some examples, the VR device (or the streaming device) may communicate, using a network interface coupled to a memory of the VR/streaming device, exchange messages to an external device, where the exchange messages are associated with the multiple available representations of the soundfield. In some examples, the VR device may receive, using an antenna coupled to the network interface, wireless signals including data packets, audio packets, video pacts, or transport protocol data associated with the multiple available representations of the soundfield. In some examples, one or more microphone arrays may capture the soundfield.

In some examples, the multiple available representations of the soundfield stored to the memory device may include a plurality of object-based representations of the soundfield, higher order ambisonic representations of the soundfield, mixed order ambisonic representations of the soundfield, a combination of object-based representations of the soundfield with higher order ambisonic representations of the soundfield, a combination of object-based representations of the soundfield with mixed order ambisonic representations of the soundfield, or a combination of mixed order representations of the soundfield with higher order ambisonic representations of the soundfield.

In some examples, one or more of the soundfield representations of the multiple available representations of the soundfield may include at least one high-resolution region and at least one lower-resolution region, and wherein the selected presentation based on the steering angle provides a greater spatial precision with respect to the at least one high-resolution region and a lesser spatial precision with respect to the lower-resolution region.

As used herein, “A and/or B” means “A or B”, or “both A and B”.

In this way, various aspects of the techniques may enable the following clauses.

Clause 1A. A device configured to reproduce a soundfield based on audio data within a vehicle, the device comprising: a memory configured to store the audio data; and processing circuitry coupled to the memory, and configured to: obtain, from an occupant monitoring system, a state of an occupant residing within a cabin of the vehicle; modify, based on the state of the occupant residing within the cabin of the vehicle, playback of the audio data within at least a portion of the cabin of the vehicle to obtain modified playback data for the audio data; and reproduce, based on the modified playback data, the soundfield.

Clause 2A. The device of clause 1A, wherein the processing circuitry, when configured to obtain the state of the occupant residing within the vehicle, is configured to obtain a state of a rear occupant residing within a rear passenger zone of the cabin of the vehicle.

Clause 3A. The device of any combination of clauses 1A and 2A, wherein the state of the occupant residing within the vehicle includes a sleeping state of the occupant residing within a rear passenger zone of the cabin of the vehicle.

Clause 4A. The device of clause 3A, wherein the processing circuitry, when configured to modify the playback of the audio data, is configured to mute, responsive to the sleeping state indicating that the occupant residing within the rear passenger zone of the cabin of the vehicle is sleeping, audio playback in the rear passenger zone of the cabin of the vehicle.

Clause 5A. The device of any combination of clauses 3A and 4A, wherein the processing circuitry, when configured to modify the playback of the audio data, is configured to perform, responsive to the sleeping state indicating that the occupant residing within the rear passenger zone of the cabin of the vehicle is sleeping, active noise cancellation with respect to the rear passenger zone of the cabin of the vehicle that includes modifying the audio data prior to playback within the rear passenger zone to limit the soundfield from being reproduced in the rear passenger zone.

Clause 6A. The device of any combination of clauses 3A-5A, wherein the processing circuitry, when configured to modify the playback of the audio data, is configured to replace, responsive to the sleeping state indicating that the occupant residing within the rear passenger zone of the cabin of the vehicle is sleeping, portions of the audio data with soothing audio data to facilitate the sleeping state of the occupant residing within the rear passenger zone of the cabin of the vehicle.

Clause 7A. The device of any combination of clauses 2A-6A, wherein the processing circuitry is further configured to alert, responsive to detecting the state of the occupant in the rear passenger zone of the cabin of the vehicle, an operator of the vehicle that the occupant in the rear passenger zone of the cabin of the vehicle is a child.

Clause 8A. The device of any combination of clauses 2A-7A, wherein the processing circuitry is further configured to adjust, responsive to detecting the state of the occupant in the rear passenger zone of the cabin of the vehicle, a heating, air conditioning, and ventilation setting for the rear passenger zone of the cabin of the vehicle.

Clause 9A. The device of any combination of clauses 1A-8A, wherein the processing circuitry is further configured to: obtain an operational state of the vehicle; and perform, based on the state of the occupant residing within the vehicle and the operational state of the vehicle, a safety action to facilitate safety with respect to the occupant residing within the vehicle.

Clause 10A. The device of clause 9A, wherein the operational state of the vehicle indicates that the vehicle is locked without access to heating, air conditioning, and ventilation, wherein the state of the occupant residing within the vehicle indicates that the occupant is still residing within the vehicle, and wherein the processing circuitry, when configured to perform the safety action, initiates, responsive to the operational state of the vehicle indicating that the occupant is still residing within the vehicle and the state of the occupant indicating that the occupant is still residing within the vehicle, a phone call to one or more of an owner of the vehicle, an operator of the vehicle, a preferred contact for the vehicle, and emergency services.

Clause 11A. The device of any combination of clauses 9A and 10A, wherein the operational state of the vehicle indicates that the vehicle is locked without access to heating, air conditioning, and ventilation, wherein the state of the occupant residing within the vehicle indicates that the occupant is still residing within the vehicle, and wherein the processing circuitry, when configured to perform the safety action, initiates, responsive to the operational state of the vehicle indicating that the occupant is still residing within the vehicle and the state of the occupant indicating that the occupant is still residing within the vehicle, a text message to one or more of an owner of the vehicle, an operator of the vehicle, a preferred contact for the vehicle, and emergency services.

Clause 12A. The device of any combination of clauses 9A-11A, wherein the operational state of the vehicle indicates that the vehicle is locked without access to heating, air conditioning, and ventilation, wherein the state of the occupant residing within the vehicle indicates that the occupant is still residing within the vehicle, and wherein the processing circuitry, when configured to perform the safety action, initiates, responsive to the operational state of the vehicle indicating that the occupant is still residing within the vehicle and the state of the occupant indicating that the occupant is still residing within the vehicle, a safety alarm to alert people nearby of the occupant within the vehicle.

Clause 13A. The device of any combination of clauses 1A-12A, wherein the processing circuitry is coupled to one or more loudspeakers, wherein the processing circuitry, when configured to reproduce the soundfield, is configured to output the modified playback data to the one or more loudspeakers, and wherein the one or more loudspeakers are configured to reproduce, based on the modified playback data and the audio data, the soundfield.

Clause 14A. The device of any combination of clauses 1A-13A, wherein the device comprises a vehicle head unit.

Clause 15A. A method of reproducing a soundfield based on audio data within a vehicle, the method comprising: obtaining, from an occupant monitoring system, a state of an occupant residing within a cabin of the vehicle; modifying, based on the state of the occupant residing within a cabin of the vehicle, playback of the audio data within at least a portion of the cabin of the vehicle to obtain modified playback data for the audio data; and reproducing, based on the modified playback data, the soundfield.

Clause 16A. The method of clause 15A, wherein obtaining the state of the occupant residing within the vehicle comprises obtaining a state of a rear occupant residing within a rear passenger zone of the cabin of the vehicle.

Clause 17A. The method of any combination of clauses 15A and 16A, wherein the state of the occupant residing within the vehicle includes a sleeping state of the occupant residing within a rear passenger zone of the cabin of the vehicle.

Clause 18A. The method of clause 17A, wherein modifying the playback of the audio data comprises muting, responsive to the sleeping state indicating that the occupant residing within the rear passenger zone of the cabin of the vehicle is sleeping, audio playback in the rear passenger zone of the cabin of the vehicle.

Clause 19A. The method of any combination of clauses 17A and 18A, wherein modifying the playback of the audio data comprises performing, responsive to the sleeping state indicating that the occupant residing within the rear passenger zone of the cabin of the vehicle is sleeping, active noise cancellation with respect to the rear passenger zone of the cabin of the vehicle that includes modifying the audio data prior to playback within the rear passenger zone to limit the soundfield from being reproduced in the rear passenger zone.

Clause 20A. The method of any combination of clauses 17A-19A, wherein modifying the playback of the audio data comprises replacing, responsive to the sleeping state indicating that the occupant residing within the rear passenger zone of the cabin of the vehicle is sleeping, portions of the audio data with soothing audio data to facilitate the sleeping state of the occupant residing within the rear passenger zone of the cabin of the vehicle.

Clause 21A. The method of any combination of clauses 16A-20A, further comprising alerting, responsive to detecting the state of the occupant in the rear passenger zone of the cabin of the vehicle, an operator of the vehicle that the occupant in the rear passenger zone of the cabin of the vehicle is a child.

Clause 22A. The method of any combination of clauses 16A-21A, further comprising adjusting, responsive to detecting the state of the occupant in the rear passenger zone of the cabin of the vehicle, a heating, air conditioning, and ventilation setting for the rear passenger zone of the cabin of the vehicle.

Clause 23A. The method of any combination of clauses 15A-22A, further comprising: obtaining an operational state of the vehicle; and performing, based on the state of the occupant residing within the vehicle and the operational state of the vehicle, a safety action to facilitate safety with respect to the occupant residing within the vehicle.

Clause 24A. The method of example 23A, wherein the operational state of the vehicle indicates that the vehicle is locked without access to heating, air conditioning, and ventilation, wherein the state of the occupant residing within the vehicle indicates that the occupant is still residing within the vehicle, and wherein performing the safety action comprises initiating, responsive to the operational state of the vehicle indicating that the occupant is still residing within the vehicle and the state of the occupant indicating that the occupant is still residing within the vehicle, a phone call to one or more of an owner of the vehicle, an operator of the vehicle, a preferred contact for the vehicle, and emergency services.

Clause 25A. The method of any combination of clauses 23A and 24A, wherein the operational state of the vehicle indicates that the vehicle is locked without access to heating, air conditioning, and ventilation, wherein the state of the occupant residing within the vehicle indicates that the occupant is still residing within the vehicle, and wherein performing the safety action comprises initiating, responsive to the operational state of the vehicle indicating that the occupant is still residing within the vehicle and the state of the occupant indicating that the occupant is still residing within the vehicle, a text message to one or more of an owner of the vehicle, an operator of the vehicle, a preferred contact for the vehicle, and emergency services.

Clause 26A. The method of any combination of clauses 23A-25A, wherein the operational state of the vehicle indicates that the vehicle is locked without access to heating, air conditioning, and ventilation, wherein the state of the occupant residing within the vehicle indicates that the occupant is still residing within the vehicle, and wherein performing the safety action comprises initiating, responsive to the operational state of the vehicle indicating that the occupant is still residing within the vehicle and the state of the occupant indicating that the occupant is still residing within the vehicle, a safety alarm to alert people nearby of the occupant within the vehicle.

Clause 27A. The method of any combination of clauses 15A-26A, wherein reproducing the soundfield comprises outputting the modified playback data to one or more loudspeakers.

Clause 28A. The method of any combination of clauses 15A-27A, wherein the device comprises a vehicle head unit.

Clause 29A. A non-transitory computer-readable storage medium having instructions stored thereon that, when executed, cause one or more processors of a vehicle headunit to: obtain, from an occupant monitoring system, a state of an occupant residing within a cabin of a vehicle including the vehicle headunit; modify, based on the state of the occupant residing within the cabin of the vehicle, playback of audio data representative of a soundfield within at least a portion of the cabin of the vehicle to obtain modified playback data for the audio data; and reproducing, based on the modified playback data and the audio data, the soundfield.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable gate arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules configured for encoding and decoding, or incorporated in a combined codec. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a codec hardware unit or provided by a collection of interoperative hardware units, including one or more processors as described above, in conjunction with suitable software and/or firmware.

Various examples have been described. These and other examples are within the scope of the following claims.

What is claimed is:

1. A device configured to reproduce a soundfield based on audio data within a vehicle, the device comprising:
a memory configured to store the audio data; and
processing circuitry coupled to the memory, and configured to:
obtain, from an occupant monitoring system, a state of an occupant residing within a cabin of the vehicle;
modify, based on the state of the occupant residing within the cabin of the vehicle, playback of the audio data within at least a portion of the cabin of the vehicle to obtain modified playback data for the audio data; and
reproduce, based on the modified playback data, the soundfield.

2. The device of claim 1, wherein the processing circuitry, when configured to obtain the state of the occupant residing within the vehicle, is configured to obtain a state of a rear occupant residing within a rear passenger zone of the cabin of the vehicle.

3. The device of claim 1, wherein the state of the occupant residing within the cabin of the vehicle includes a sleeping state of the occupant residing within a rear passenger zone of the cabin of the vehicle.

4. The device of claim 3, wherein the processing circuitry, when configured to modify the playback of the audio data, is configured to mute, responsive to the sleeping state indicating that the occupant residing within the rear passenger zone of the cabin of the vehicle is sleeping, audio playback in the rear passenger zone of the cabin of the vehicle.

5. The device of claim 3, wherein the processing circuitry, when configured to modify the playback of the audio data, is configured to perform, responsive to the sleeping state indicating that the occupant residing within the rear passenger zone of the cabin of the vehicle is sleeping, active noise cancellation with respect to the rear passenger zone of the cabin of the vehicle that includes modifying the audio data prior to playback within the rear passenger zone to limit the soundfield from being reproduced in the rear passenger zone.

6. The device of claim 3, wherein the processing circuitry, when configured to modify the playback of the audio data, is configured to replace, responsive to the sleeping state indicating that the occupant residing within the rear passenger zone of the cabin of the vehicle is sleeping, portions of the audio data with soothing audio data to facilitate the sleeping state of the occupant residing within the rear passenger zone of the cabin of the vehicle.

7. The device of claim 2, wherein the processing circuitry is further configured to alert, responsive to detecting the state of the occupant in the rear passenger zone of the cabin of the vehicle, an operator of the vehicle that the occupant in the rear passenger zone of the cabin of the vehicle is a child.

8. The device of claim 2, wherein the processing circuitry is further configured to adjust, responsive to detecting the state of the occupant in the rear passenger zone of the cabin of the vehicle, a heating, air conditioning, and ventilation setting for the rear passenger zone of the cabin of the vehicle.

9. The device of claim 1, wherein the processing circuitry is further configured to:
obtain an operational state of the vehicle; and
perform, based on the state of the occupant residing within the vehicle and the operational state of the vehicle, a safety action to facilitate safety with respect to the occupant residing within the vehicle.

10. The device of claim 9,
wherein the operational state of the vehicle indicates that the vehicle is locked without access to heating, air conditioning, and ventilation,
wherein the state of the occupant residing within the vehicle indicates that the occupant is still residing within the vehicle, and
wherein the processing circuitry, when configured to perform the safety action, initiates, responsive to the operational state of the vehicle indicating that the occupant is still residing within the vehicle and the state of the occupant indicating that the occupant is still residing within the vehicle, a phone call to one or more of an owner of the vehicle, an operator of the vehicle, a preferred contact for the vehicle, and emergency services.

11. The device of claim 9,
wherein the operational state of the vehicle indicates that the vehicle is locked without access to heating, air conditioning, and ventilation,
wherein the state of the occupant residing within the vehicle indicates that the occupant is still residing within the vehicle, and
wherein the processing circuitry, when configured to perform the safety action, initiates, responsive to the operational state of the vehicle indicating that the occupant is still residing within the vehicle and the state of the occupant indicating that the occupant is still residing within the vehicle, a text message to one or more of an owner of the vehicle, an operator of the vehicle, a preferred contact for the vehicle, and emergency services.

12. The device of claim 9,
wherein the operational state of the vehicle indicates that the vehicle is locked without access to heating, air conditioning, and ventilation,
wherein the state of the occupant residing within the vehicle indicates that the occupant is still residing within the vehicle, and
wherein the processing circuitry, when configured to perform the safety action, initiates, responsive to the operational state of the vehicle indicating that the occupant is still residing within the vehicle and the state of the occupant indicating that the occupant is still residing within the vehicle, a safety alarm to alert people nearby of the occupant within the vehicle.

13. The device of claim 1,
wherein the processing circuitry is coupled to one or more loudspeakers,
wherein the processing circuitry, when configured to reproduce the soundfield, is configured to output the modified playback data to the one or more loudspeakers, and
wherein the one or more loudspeakers are configured to reproduce, based on the modified playback data and the audio data, the soundfield.

14. The device of claim 1, wherein the device comprises a vehicle head unit.

15. A method of reproducing a soundfield based on audio data within a vehicle, the method comprising:
obtaining, from an occupant monitoring system, a state of an occupant residing within a cabin of the vehicle;
modifying, based on the state of the occupant residing within a cabin of the vehicle, playback of the audio data within at least a portion of the cabin of the vehicle to obtain modified playback data for the audio data; and
reproducing, based on the modified playback data, the soundfield.

16. The method of claim 15, wherein obtaining the state of the occupant residing within the cabin of the vehicle comprises obtaining a state of a rear occupant residing within a rear passenger zone of the cabin of the vehicle.

17. The method of claim 15, wherein the state of the occupant residing within the vehicle includes a sleeping state of the occupant residing within a rear passenger zone of the cabin of the vehicle.

18. The method of claim 17, wherein modifying the playback of the audio data comprises muting, responsive to the sleeping state indicating that the occupant residing within the rear passenger zone of the cabin of the vehicle is sleeping, audio playback in the rear passenger zone of the cabin of the vehicle.

19. The method of claim 17, wherein modifying the playback of the audio data comprises performing, responsive to the sleeping state indicating that the occupant residing within the rear passenger zone of the cabin of the vehicle is sleeping, active noise cancellation with respect to the rear passenger zone of the cabin of the vehicle that includes modifying the audio data prior to playback within the rear passenger zone to limit the soundfield from being reproduced in the rear passenger zone.

20. The method of claim 17, wherein modifying the playback of the audio data comprises replacing, responsive to the sleeping state indicating that the occupant residing within the rear passenger zone of the cabin of the vehicle is sleeping, portions of the audio data with soothing audio data to facilitate the sleeping state of the occupant residing within the rear passenger zone of the cabin of the vehicle.

21. The method of claim 16, further comprising alerting, responsive to detecting the state of the occupant in the rear passenger zone of the cabin of the vehicle, an operator of the vehicle that the occupant in the rear passenger zone of the cabin of the vehicle is a child.

22. The method of claim 16, further comprising adjusting, responsive to detecting the state of the occupant in the rear passenger zone of the cabin of the vehicle, a heating, air conditioning, and ventilation setting for the rear passenger zone of the cabin of the vehicle.

23. The method of claim 15, further comprising:
obtaining an operational state of the vehicle; and
performing, based on the state of the occupant residing within the vehicle and the operational state of the vehicle, a safety action to facilitate safety with respect to the occupant residing within the vehicle.

24. The method of claim 23,
wherein the operational state of the vehicle indicates that the vehicle is locked without access to heating, air conditioning, and ventilation,
wherein the state of the occupant residing within the vehicle indicates that the occupant is still residing within the vehicle, and
wherein performing the safety action comprises initiating, responsive to the operational state of the vehicle indicating that the occupant is still residing within the vehicle and the state of the occupant indicating that the occupant is still residing within the vehicle, a phone call to one or more of an owner of the vehicle, an operator of the vehicle, a preferred contact for the vehicle, and emergency services.

25. The method of claim 23,
wherein the operational state of the vehicle indicates that the vehicle is locked without access to heating, air conditioning, and ventilation,
wherein the state of the occupant residing within the vehicle indicates that the occupant is still residing within the vehicle, and
wherein performing the safety action comprises initiating, responsive to the operational state of the vehicle indicating that the occupant is still residing within the vehicle and the state of the occupant indicating that the occupant is still residing within the vehicle, a text message to one or more of an owner of the vehicle, an operator of the vehicle, a preferred contact for the vehicle, and emergency services.

26. The method of claim 23,
wherein the operational state of the vehicle indicates that the vehicle is locked without access to heating, air conditioning, and ventilation,
wherein the state of the occupant residing within the vehicle indicates that the occupant is still residing within the vehicle, and
wherein performing the safety action comprises initiating, responsive to the operational state of the vehicle indicating that the occupant is still residing within the vehicle and the state of the occupant indicating that the occupant is still residing within the vehicle, a safety alarm to alert people nearby of the occupant within the vehicle.

27. The method of claim 15, wherein reproducing the soundfield comprises outputting the modified playback data to one or more loudspeakers.

28. The method of claim 15, wherein obtaining comprises obtaining, by a vehicle head unit, modifying comprises modifying, by the vehicle head unit, and reproducing comprises reproducing, by the vehicle head unit.

29. A non-transitory computer-readable storage medium having instructions stored thereon that, when executed, cause one or more processors of a vehicle headunit to:
obtain, from an occupant monitoring system, a state of an occupant residing within a cabin of a vehicle including the vehicle headunit;
modify, based on the state of the occupant residing within the cabin of the vehicle, playback of audio data representative of a soundfield within at least a portion of the cabin of the vehicle to obtain modified playback data for the audio data; and
reproducing, based on the modified playback data and the audio data, the soundfield.
